(19) United States
(12) Patent Application Publication
(10) Pub. No.: US 2023/0020683 A1
(43) Pub. Date: Jan. 19, 2023

(54) MAGNETIC MANIPULATION THROUGH SOLID-STATE METHOD AND APPARATUS

(71) Applicant: Siemens Healthcare Diagnostics Inc., Tarrytown, NY (US)

(72) Inventors: Philip Chia, El Cerrito, CA (US); Melaku Woldemariam, San Leandro, CA (US); Ben Blizard, Oakland, CA (US)

(73) Assignee: Siemens Healthcare Diagnostics Inc., Tarrytown, NY (US)

(21) Appl. No.: 17/757,745

(22) PCT Filed: Jan. 26, 2021
(86) PCT No.: PCT/US2021/070079

§ 371 Date: Jun. 20, 2022

Related U.S. Application Data
(60) Provisional application No. 62/969,713, filed on Feb. 4, 2020.

(51) Int. Cl. C12N 15/10; B03C 1/01; B03C 1/033; B03C 1/28; B01F 33/451

(52) U.S. Cl. CPC C12N 15/1013; B03C 1/01; B03C 1/0335; B03C 1/288; B01F 33/451; B03C 2201/18; B03C 2201/26; B01F 2101/23

(57) ABSTRACT

An apparatus and method for extracting nucleic acids such as DNA molecules from biological samples uses solid-state magnetic manipulation. A fluid sample and magnetic beads are placed in a vessel. The vessel is placed in a housing with an array of electromagnets mounted therein. The electromagnets are energized sequentially or in groups to move the magnetic beads through the fluid sample in a variety of patterns. The apparatus disclosed herein may be used as a measurement device to measure bead number density and modify magnetic patterns in order to deliver consistent dosages in bead number.

### RELATED APPLICATIONS

This patent application claims priority from U.S. Provisional Patent Application No. 62/969,713, filed Feb. 4, 2020.

### FIELD OF THE DISCLOSURE

The disclosure herein relates generally to the field of cell lysing and nucleic acid purification and isolation. More particularly, the present disclosure relates to devices and methods for mechanically mixing a magnetic microparticles in a sample fluid.

### BACKGROUND

Cell lysis and nucleic acid isolation may use magnetic beads, or microparticles, to mix solutions and separate nucleic acids from solution. When isolating DNA, the dose per assay of microparticles used for nucleic acid transfer impacts assay performance. Variation in the microparticle dose can cause less or more nucleic acid to be bound, transferred, and then released (eluted) for use in the reaction. Accurate and repeatable control of the dosing is important to assay performance, in particular repeatability. This invention could provide more consistent dosing.

Typically, mechanical means of mixing ferrous microparticles (magnetic beads) with a suspending fluid requires moving parts. This undermines instrument reliability due to wear. It also increases operating noise, as well as the risk of splattering and aerosolization of biohazardous samples.

US Published Patent Applications 2013/0217144 and 2017/0284922 describe a method and apparatus for mixing magnetic particles in a microfluidic chamber on a chip using alternating 4-pole electromagnets. As a sample fluid enters the microfluidic chamber containing ferromagnetic and superparamagnetic particles from one edge, the particles rotate and mix in a two-dimensional plane as the fluid flows through to the outlet of the chamber. Magnets are electromagnetically actuated at various strengths and frequencies to manipulate both the ferromagnetic and superparamagnetic particles. This system requires a special purpose cartridge and only works with very small sample sizes.

### SUMMARY

In embodiments, a system for manipulating magnetic beads for processes involving molecular manipulations includes a housing for receiving a vessel containing a fluid sample and a plurality of magnetic beads therein, the housing having a circumference and a height perpendicular to the circumference; a first plurality of electromagnets spaced evenly around the circumference of the housing at a height h₁; a second plurality of electromagnets spaced evenly around the circumference of the housing at a height h₂, the second plurality of electromagnets being equal in number to the first plurality of electromagnets, the electromagnets of the second plurality offset from the electromagnets of the first plurality around the circumference of the housing; and a controller for selectively energizing electromagnets of the first and second pluralities in sequence causing the magnetic beads to circulate in the vessel.

The controller may include a microcontroller and an H-bridge that may independently energize or reverse the polarity the electromagnets, individually or in groups. Both the first and second pluralities of electromagnets may include three electromagnets, in embodiments.

The system may include an optical scattering sensor for measuring magnetic bead density in a target location within the vessel and providing the measured bead density to the controller to enable modification of the selective energizing of the electromagnets. In addition or alternatively, the system may include a capacitance sensor for measuring magnetic bead density in a target location within the vessel and providing the measured bead density to the controller to enable modification of the selective energizing of the electromagnets.

In embodiments, a method of mixing magnetic beads in a fluid sample in a molecular analysis application may include depositing the fluid sample and magnetic beads within a vessel; positioning the vessel within a housing having a circumference and a height perpendicular to the circumference; disposing a first plurality of electromagnets evenly around the circumference of the housing at a height h₁, the electromagnets imparting a magnetic field within the vessel when energized; disposing a second plurality of electromagnet evenly around the circumference of the housing at a height h₂, the second plurality of electromagnets equal in number to the first plurality of electromagnets and each electromagnet of the second plurality offset from the electromagnets of the first plurality around the circumference of the housing, the electromagnets imparting a magnetic field within the vessel when energized; and selectively energizing the electromagnets of the first and second pluralities in a sequence causing the magnetic beads to circulate throughout the vessel.

The method may further include positioning electromagnets of the first plurality between electromagnets of the second plurality around the circumference of the housing.

The method may include selectively energizing by causing the magnetic beads to circulate around a vertical axis by energizing the first electromagnet; energizing the second electromagnet; energizing the third electromagnet; energizing the fourth electromagnet; energizing the fifth electromagnet; and energizing the sixth electromagnet.

In embodiments, the method may include selectively energizing by causing the magnetic beads to move up and down in the vessel by energizing the first electromagnet; energizing the fifth electromagnet; energizing the second electromagnet; energizing the sixth electromagnet; energizing the third electromagnet; and energizing the fourth electromagnet.

In embodiments, a method of drawing a sample having a quantity of magnetic beads from a sample fluid includes depositing the fluid sample and magnetic beads within a vessel; positioning the vessel within a housing having a circumference and a height perpendicular to the circumference, the housing comprising an array of electromagnets, the array comprising a first plurality of electromagnets spaced evenly around the circumference of the housing at a height h₁, and a second plurality of electromagnets spaced evenly around the circumference of the housing at a height h₂, the second plurality of electromagnets being equal in number to the first plurality of electromagnets, the electromagnets of the second plurality offset from the electromagnets of the first plurality around the circumference of the housing; selectively energizing the first and second pluralities of electromagnets in a sequence to cause the magnetic beads to circulate throughout the vessel; measuring magnetic bead density in a target location of the fluid within the vessel; modifying a sequence of energizing the first and second pluralities of electromagnets to maintain a consistent magnetic bead density in the target location; and drawing the sample from the target location of the fluid.

The method may include measuring magnetic bead density by measuring an optical scattering of the magnetic beads in the target location. In addition or alternatively, the method may include measuring a capacitance of the magnetic beads in the target location.

The sequence of selectively energizing the electromagnets may be chosen to cause the magnetic beads to rotate around the longitudinal axis of the vessel while simultaneously moving up and down within the vessel.

### BRIEF DESCRIPTION OF THE DRAWINGS

Illustrative embodiments of the disclosed technology are described in detail below with reference to the attached drawing figures, which are incorporated by reference herein and wherein:

FIG. 1 illustrates a perspective view of a solid state magnetic manipulation apparatus, in embodiments.

FIG. 2 is a side view of the apparatus of FIG. 1, in embodiments.

FIG. 3 is a top view of the apparatus of FIG. 1, in embodiments.

FIG. 4 is a bottom view of the apparatus of FIG. 1, in embodiments.

FIGS. 5A-5F illustrate a process of mixing magnetic particles in the apparatus of FIG. 1, in embodiments.

FIG. 6 is a flowchart illustrating a method of solid-state magnetic manipulation, in embodiments.

FIG. 7 is a flowchart illustrating a method of measuring magnetic particle density, in embodiments.

### DETAILED DESCRIPTION

Disclosed herein is an apparatus and method for extracting nucleic acids such as DNA molecules from biological samples. In embodiments, no moving parts are used to vortex ferrous microparticles, which reduces the risk of splattering of the sample liquid and machine wear. It also reduces operating noise when compared to the traditional mechanical based mixing. In addition, this invention can utilize ferrous microparticles as a stirrer to mix heterogeneous liquids in low-cost and standard size tubes without the need of pipettes, microfluidic chips/channels, centrifuges, vortexers, or other mechanical devices and niche consumables. In embodiments, apparatus disclosed herein may be used as a measurement device to measure bead number density and modify magnetic patterns in order to deliver (closed-loop) consistent dosages in bead number. The terms magnetic beads, magnetic particles, nanoparticles and ferrofluid are used interchangeably herein.

In embodiments, magnetic beads may be magnetic micron- or nano-particles with a surface modification which binds target nucleic acids released during a sample preparation process of biological specimens. In the systems and methods described herein, a magnetic field is the only driving force for sample and/or magnetic bead handling.

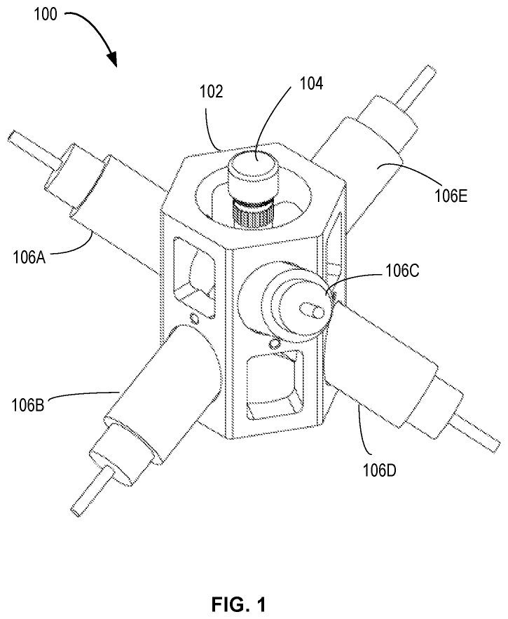

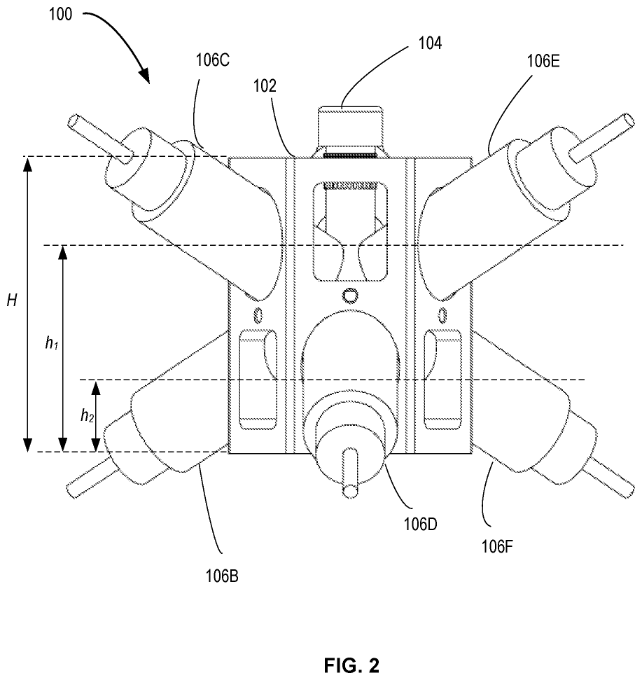

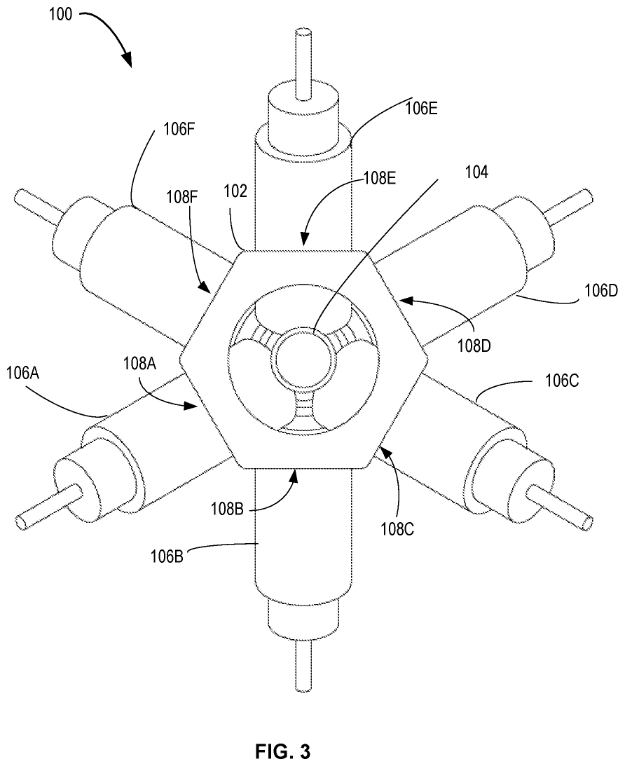

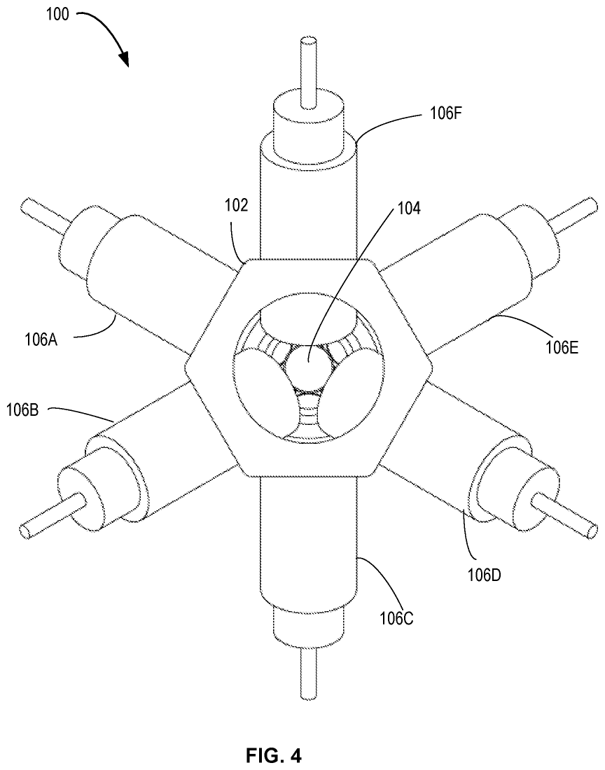

FIG. 1 illustrates a perspective view of an exemplary embodiment of an apparatus 100 for mixing fluids and magnetic particles in low cost and commercially available tube and vial consumables. FIG. 2 is a side view, FIG. 3 is a top view and FIG. 4 is a bottom view of the apparatus of FIG. 1, in embodiments. FIGS. 1-4 are best viewed together in the following description.

The apparatus 100 is comprised of a housing 102 for retaining a fluid sample tube 104. In embodiments, tube 104 may contain a 2 mL fluid sample but apparatus 100 is not limited to any particular sample size. As shown in FIGS. 1-4, housing 102 is generally formed as a cylinder with six sides 108, and has a circumference and an overall height H. Each side 108 (FIG. 3) has an upper aperture in a horizontal plane at a height h₁ and a lower aperture in a horizontal plane at a height h₂.

An array of six electromagnets is positioned radially around the circumference of housing 102. Although the electromagnets are shown with a central axis that is angled relative to the sides of the housing, this is not required. Three electromagnets 106A, 106C and 106E are arranged radially in the horizontal plane at a height hi from the bottom of housing 102 as shown in FIG. 2. In the horizontal plane at height hi, electromagnets 106A, 106C and 106E are inserted through apertures in sides 108A, 108C and 108E respectively, to a position close to tube 104. Apertures in sides 108B, 108D and 108F are open.

Three electromagnets 106B, 106D and 106F are arranged radially in the horizontal plane at a height h₂ from the bottom of housing 102. In the horizontal plane at height h₂, electromagnets 106B, 106D and 106F are inserted into apertures in sides 108B, 108D and 108F, respectively, of housing 102. These are sides 108 that have open apertures in the horizontal plane at height h₁. Electromagnets 106 alternate around the circumference of housing 102 such that electromagnet 106B, for example, is positioned between electromagnets 106A and 106C.

Electromagnets 106 may be energized sequentially or in groups to induce a wide variety of movements to magnetic beads in tube 104. Magnetic beads may be caused to spin from the base of the tube 104 to the top of the tube in a helical (three-dimensional vortex) motion. Other patterns of movement are contemplated, such as moving up and down vertically along the longitudinal axis of the tube or around the circumference of tube 104 in a horizontal plane, for example.

Electromagnets 106 may be, for example, 12-24VDC electromagnets. A controller (not shown), may be used to switch electromagnets 106 ON and OFF. An “ON” state indicates that the electromagnet is generating a magnetic field and an “OFF” state means that it is not generating a magnetic field. In embodiments, electromagnets 106 may be controlled with any programmable control device, such as an Arduino Mega (2560) microcontroller and an H-Bridge (L298N) with flyback diodes. The pulsed signal (˜5Hz) generated from the Arduino Mega microcontroller triggers the H-Bridge to turn on as well as reverse the polarity of an individual electromagnet 106.

Although housing 102 is shown and discussed with a specific arrangement of sides and apertures, this is for purposes of illustration only. Housing 102 may have more or fewer sides. Electromagnets may be arranged in more than two horizontal planes and the heights of the planes may be evenly spaced along overall height H or have different spacing. A horizontal plane may have any number of electromagnets. Further, sides 108 may not include an aperture where no electromagnet is inserted. Further, a cross-section of housing 102 may be circular instead of having distinct sides.

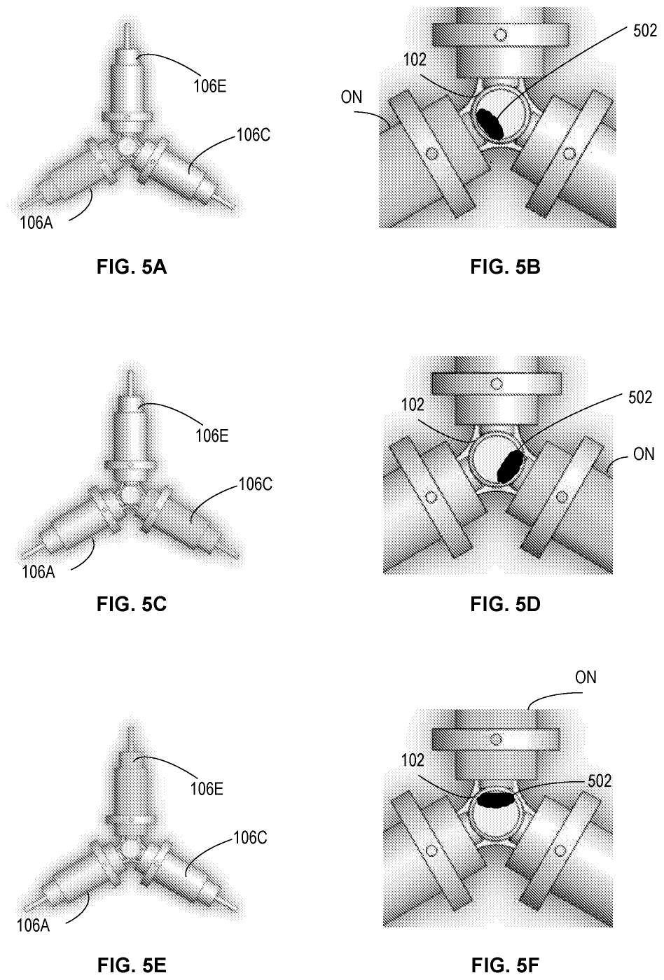

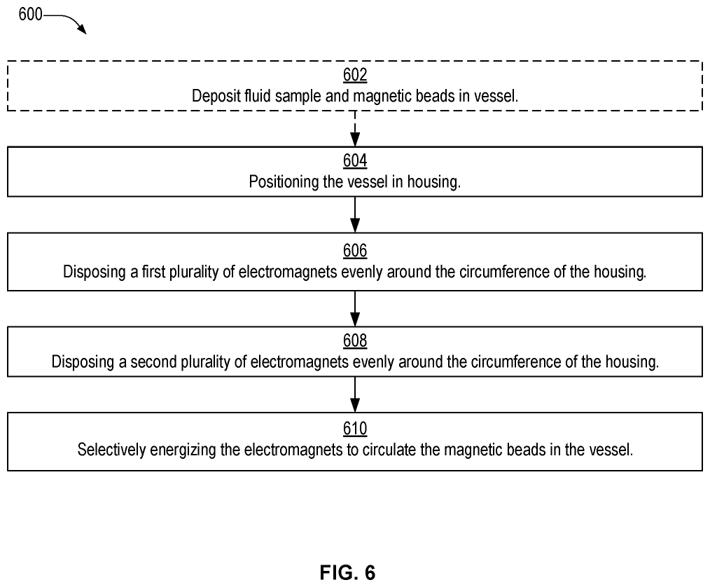

Operation of apparatus 100 to perform a solid-state mixing operation of magnetic particles will now be described. FIGS. 5A-5F illustrate a movement of magnetic particles while FIG. 6 is a flowchart illustrating the method, in embodiments. FIGS. 5A-5F and 6 are best viewed together in the following discussion.

FIG. 6 is a flowchart of a method 600 of solid-state magnetic manipulation using apparatus 100 of FIG. 1. Not all steps need be practiced in the order described below, nor be utilized at all, depending upon the embodiment.

Step 602 includes depositing a fluid sample and magnetic beads in a vessel. In an example of step 602, a fluid sample may be any fluid containing a nucleic acid for analysis. Magnetic beads may be microparticles or nanoparticles, any ferromagnetic particle or superparamagnetic particle. The vessel may be a low cost and commercially available tube or vial consumables such as a 2mL tube. In embodiments, method 600 includes step 602 however, method 600 is not limited to placing the fluid sample and magnetic beads in a separate vessel.

Step 604 includes positioning a vessel in a housing. In an example of step 602, a vessel 104 containing a fluid sample and magnetic beads is placed in a housing 102.

Step 606 includes disposing a first plurality of electromagnets evenly around the circumference of the housing 102 at height h₁. In an example of step 606, housing 102 has six sides. Three sides have electromagnets disposed thereon at height h₁ while the other three sides have no electromagnets at height h₁, as shown in FIGS. 1-4.

Step 608 includes disposing a second plurality of electromagnets evenly around the circumference of the housing 102 at height h₂. In an example of step 608, housing 102 has six sides. The three sides with no electromagnets in step 606 at height h₁ have electromagnets 106B, 106D and 106F disposed thereon at height h₂ while the other three sides have no electromagnets at height h₂, as shown in FIGS. 1-4.

Although two pluralities of three electromagnets are discussed herein, any number of electromagnets may be used as long as they are evenly spaced around a circumference of housing 102 in an arrangement that alternates electromagnets at height h₁ and h₂.

Step 610 includes selectively energizing the electromagnets of the first and second pluralities in a sequence causing the magnetic beads to circulate throughout the vessel. In an example of step 610, the electromagnetic configuration described herein allows the beads to gradually spiral from the base of the tube to the top of the tube and back down. As shown in FIGS. 5A-5F, one electromagnet is energized at a time to move magnetic beads through the interior of vessel 104. As shown in FIG. 5A, electromagnet 106A on the bottom left is turned ON. FIG. 5B shows a close-up view of the center of FIG. 5A where magnetic beads 502 have been drawn to electromagnet 106A. Although FIGS. 5A-5F are discussed with reference to electromagnets 106A, 106C and 106E, the discussion herein also applies to electromagnets 106B, 106D and 106F.

Continuing with step 610, in FIGS. 5C and 5D, electromagnet 106A is de-energized while electromagnet 106C is energized so that magnetic beads 502 are drawn to electromagnet 106C. In FIGS. 5E and 5F, electromagnet 106C is de-energized while electromagnet 106E is energized so that magnetic beads 502 are drawn to electromagnet 106E. Electromagnets around the circumference of housing 102 are energized in sequence (akin to the operating principle of a stepper motor) thus enabling the magnetic beads to circulate in vessel 104 one step at a time. The speed of magnetic bead circulation is dependent on the rate that the electromagnets sequentially turn on and off

Embodiments described herein use no moving parts to vortex ferrous microparticles which reduces the risk of splattering of the sample liquid, reduces wear as well as reduces operating noise when compared to the traditional mechanical based mixing. In addition, apparatus 100 may utilize ferrous microparticles as a stirrer to mix heterogeneous liquids in standard size tubes without the need of pipettes, microfluidic chips/channels, centrifuges, vortexers, or other mechanical devices and niche consumables.

Further, apparatus 100 is completely solid-state, requires little to no maintenance, and uses no tubing, valves, pumps, and other fluidic devices that will wear and/or clog from use. Apparatus 100 may also be used as a miniaturized stir plate with the magnetic particles placed in the fluid in lieu of a conventional stir bar.

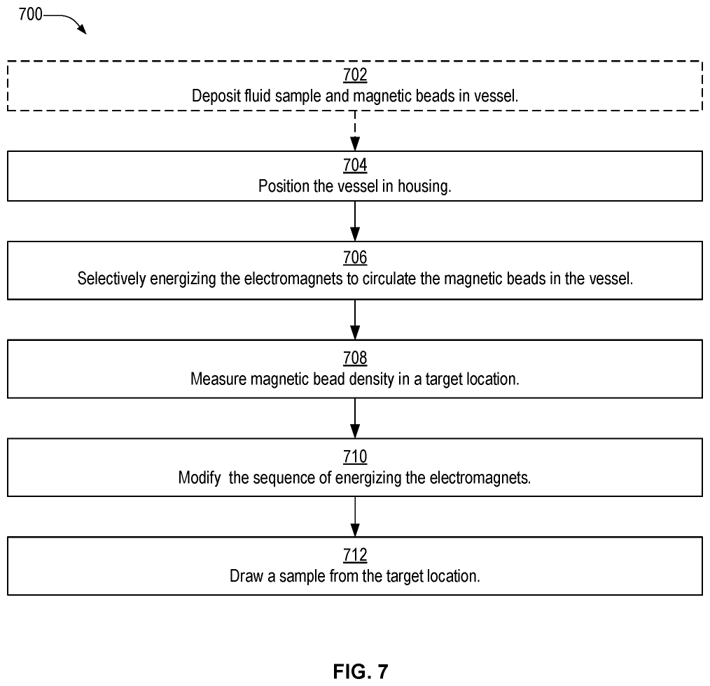

FIG. 7 is a flowchart illustrating a method 700 of measuring magnetic particle density using apparatus 100, in embodiments. Not all steps need be practiced in the order described below, nor be utilized at all, depending upon the embodiment. In addition to the apparatus discussed in connection with FIGS. 1-4, apparatus 100 used for the method of FIG. 7 would also include a physical measurement device such as camera having a light source and aperture.

Step 702 includes depositing a fluid sample and magnetic beads in a vessel. In an example of step 702, a fluid sample may be any fluid containing a nucleic acid for analysis. Magnetic beads may be microparticles or nanoparticles, any ferromagnetic particle or superparamagnetic particle. The vessel may be a low cost and commercially available tube or vial consumables such as a 2 mL tube. In embodiments, method 700 includes step 702 however, method 700 is not limited to placing the fluid sample and magnetic beads in a separate vessel.

Step 704 includes positioning a vessel in a housing. In an example of step 702, a vessel 104 containing a fluid sample and magnetic beads is placed in a housing 102.

Step 706 includes disposing a first plurality of electromagnets evenly around the circumference of the housing 102 at height h₁. In an example of step 706, housing 102 has six sides. Three sides have electromagnets disposed thereon at height h₁ while the other three sides have no electromagnets at height h₁, as shown in FIGS. 1-4.

Step 706 also includes disposing a second plurality of electromagnets evenly around the circumference of the housing 102 at height h₂. The three sides with no electromagnets in step 706 at height hi have electromagnets 106B, 106D and 106F disposed thereon at height h₂, as shown in FIGS. 1-4.

Step 708 includes measuring magnetic bead density in a target location. In an example of step 708, a measuring device such as a camera, is positioned to measure magnetic bead density in vessel 104 while magnetic beads are being mixed in the vessel. In embodiments, measurement of bead number density in a target area may include optical scattering or capacitance. This measurement is fed back to the electromagnet control system to do a closed loop control of magnetic bead density so that a uniform bead dose may be sampled.

Step 710 includes modifying the sequence of energizing the electromagnets. In an example of step 710, this modification may maintain a uniform distribution of magnetic beads in the target location over time based on feedback from step 708. This modification may include changing the speed of energizing electromagnets, or the order in which they are energized. The sequence may mix the magnetic beads horizontally and/or vertically vessel 104, or switch between the two.

Step 712 includes drawing a sample from the target location. In an example of step 712, the modified sequence of step 710 changes the concentration of magnetic beads in the target location so that a known quantity of nucleic acid may be removed from vessel 104.

Changes may be made to embodiments described herein. Implementations of apparatus 100 may include physical rocking, shaking and inversions. Ultrasonic vibrations may be used to mix beads or generate a standing wave in appropriate acoustic environment.

Embodiments disclosed herein may be used, for example, in molecular diagnostics in nucleic acid extraction for real time PCR as well as an alternative to traditional mixing methods (e.g. pipette mixing, stir plate, etc.).

Many changes in the details, materials, and arrangement of parts and steps, herein described and illustrated, can be made by those skilled in the art in light of teachings contained hereinabove. It will be understood that certain features and sub-combinations are of utility and may be employed without reference to other features and sub combinations and are contemplated within the scope of the claims. Accordingly, it will be understood that the following claims are not to be limited to the embodiments disclosed herein and can include practices other than those specifically described, and are to be interpreted as broadly as allowed under the law. Additionally, not all steps listed in the various figures need be carried out in the specific order described.

We claim:

1. A system for manipulating magnetic beads for processes involving molecular manipulations, comprising:
a housing receiving a vessel containing a fluid sample and a plurality of magnetic beads therein, the housing having a circumference and a height perpendicular to the circumference;
a first plurality of electromagnets spaced evenly around the circumference of the housing at a height h₁;
a second plurality of electromagnets spaced evenly around the circumference of the housing at a height h₂, the second plurality of electromagnets being equal in number to the first plurality of electromagnets, the electromagnets of the second plurality offset from the electromagnets of the first plurality around the circumference of the housing; and
a controller for selectively energizing electromagnets of the first and second pluralities in sequence causing the magnetic beads to circulate in the vessel.

2. The system of claim 1, wherein the controller further comprises a microcontroller and an H-bridge.

3. The system of claim 2, wherein the microcontroller generates a signal for independently turning the electromagnets on and off individually or in groups.

4. The system of claim 2, wherein the microcontroller reverses the polarity of the electromagnets.

5. The system of claim 1, wherein the first plurality further comprises three electromagnets and the second plurality further comprises three electromagnets.

6. The system of claim 1, wherein the housing holds the electromagnets in position around the circumference of the vessel when the vessel is received within the housing.

7. The system of claim 1, further comprising an optical scattering sensor for measuring magnetic bead density in a target location within the vessel and providing the measured bead density to the controller to enable modification of the sequence of selective energizing of the electromagnets.

8. The system of claim 1, further comprising a capacitance sensor for measuring magnetic bead density in a target location within the vessel and providing the measured bead density to the controller to enable modification of the selective energizing of the electromagnets.

9. A method of mixing magnetic beads in a fluid sample in a molecular analysis application, comprising:
depositing the fluid sample and magnetic beads within a vessel;
positioning the vessel within a housing having a circumference and a height perpendicular to the circumference;
disposing a first plurality of electromagnets evenly around the circumference of the housing at a height ₁, the electromagnets imparting a magnetic field within the vessel when energized;
disposing a second plurality of electromagnet evenly around the circumference of the housing at a height h₂, the second plurality of electromagnets equal in number to the first plurality of electromagnets and each electromagnet of the second plurality offset from the electromagnets of the first plurality around the circumference of the housing, the electromagnets imparting a magnetic field within the vessel when energized; and
selectively energizing the electromagnets of the first and second pluralities in a sequence causing the magnetic beads to circulate throughout the vessel.

10. The method of claim 9, wherein the first plurality further comprises first, second and third electromagnets and the second plurality further comprises fourth, fifth and sixth electromagnets wherein the fourth electromagnet is positioned between the first and third electromagnets.

11. The method of claim 10, wherein selectively energizing further comprises causing the magnetic beads to circulate around a vertical axis by:
energizing the first electromagnet;
energizing the second electromagnet;
energizing the third electromagnet;
energizing the fourth electromagnet;
energizing the fifth electromagnet; and
energizing the sixth electromagnet.

12. The method of claim 10, wherein selectively energizing further comprises causing the magnetic beads to move up and down in the vessel by:
energizing the first electromagnet;
energizing the fifth electromagnet;
energizing the second electromagnet;
energizing the sixth electromagnet;
energizing the third electromagnet; and
energizing the fourth electromagnet.

13. A method of drawing a sample having a quantity of magnetic beads from a sample fluid, comprising:
depositing the fluid sample and magnetic beads within a vessel;
positioning the vessel within a housing having a circumference and a height perpendicular to the circumference, the housing comprising an array of electromagnets, the array comprising a first plurality of electromagnets spaced evenly around the circumference of the housing at a height h₁, and a second plurality of electromagnets spaced evenly around the circumference of the housing at a height h₂, the second plurality of electromagnets being equal in number to the first plurality of electromagnets, the electromagnets of the second plurality offset from the electromagnets of the first plurality around the circumference of the housing;
selectively energizing the first and second pluralities of electromagnets in a sequence to cause the magnetic beads to circulate throughout the vessel;
measuring magnetic bead density in a target location of the fluid sample within the vessel;
modifying a sequence of energizing the first and second pluralities of electromagnets to maintain a consistent magnetic bead density in the target location; and
drawing the sample from the target location of the fluid sample.

14. The method of claim 13, wherein measuring magnetic bead density further comprises measuring an optical scattering of the magnetic beads in the target location.

15. The method of claim 13, wherein measuring magnetic bead density further comprises measuring a capacitance of the magnetic beads in the target location.

16. The method of claim 13, wherein the sequence of selectively energizing the electromagnets is chosen to cause the magnetic beads to rotate around the longitudinal axis of the vessel while simultaneously moving up and down within the vessel.
